(19) United States
(12) Patent Application Publication
(10) Pub. No.: US 2005/0202555 A1
(43) Pub. Date: Sep. 15, 2005

(54) CELL DETECTION CHIP AND FABRICATING METHOD THEREOF AND CELL DETECTING METHOD

(76) Inventor: Yu-Jie Zhao, Shenyang City (CN)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 10/908,920

(22) Filed: Jun. 1, 2005

Related U.S. Application Data
(62) Division of application No. 10/709,413, filed on May 4, 2004.

(30) Foreign Application Priority Data
Dec. 22, 2003 (TW) 92136376

(51) Int. Cl. C12Q 1/68; C12M 1/34

(52) U.S. Cl. 435/287.2

(57) ABSTRACT

A microarray detection chip is described. The detection chip includes several probes immobilized on a matrix, and these probes have affinity with corresponding specific molecules on a cell membrane surface. Since these probes are correlated with the specific molecules on the cell membrane surface, this microarray detection chip can be used to recognize different types of the cells.

### CROSS-REFERENCE TO RELATED APPLICATION

This application is a divisional of a prior application Ser. No. 10/709,413, filed May 4, 2004, which claims the priority benefit of Taiwan application serial no. 92136376, filed on Dec. 22, 2003.

### BACKGROUND OF THE INVENTION

1. Field of the Invention

The present invention relates generally to a biological detection device and fabrication method thereof, and more particularly, to cell detection chip and fabrication method thereof and cell detecting method.

2. Description of the Related Art

Biotechnology industry has been expanding rapidly worldwide. As a technology based on studies of biological sciences and capable of affecting the quality of human life, biotechnology is so broad in its scope that it is correlated with genetic engineering, aquaculture, pharmaceutics, medical treatment, health food, and health care.

Biochip development is one of important field in biotechnology. Biochips are detection devices minimized based on principles of molecular biology, genetic informatics and analytical chemistry. With respect to detection devices based on conventional bio-analysis, biochips have advantages such as lower consumption of detection agent, faster detection rate, lower operation cost, and higher detection accuracy. Generally, biochips can be classified as two types: sensing chip and processing chip. In view of the development of biochips, processing chip is the type of ultimate target while sensing chip is the type of current focus on the chip developing market.

A sensing chip can catch tens or even hundreds of bio-molecules as microarrays on a small matrix (for example, glass). Hence, this type of biochip is also called as microarray chip. The sensing chip is used for detection mainly based on the specificity between the probes on the matrix and the specific component in the sample to be detected. For example, on a DNA chip of a sensing chip, the probes consist of different deoxyribonucleotide arrays. When the deoxyribonucleotide array contained in a biological sample (for example, from a patient) is complementary to that of a probe, the sample is “reactive” to the probe, while such reactivity has a special significance for the detection process.

On the other hand, when a person suffers from a serious disease, such as malignancy, certain protein with specific characteristics generated due to genetic mutation will appear on different regions of pathologically changed cells, and normal cells and pathologically changed cells exist concurrently in a human body. Thus, cytology detection is very important for diagnosis and treatment of malignancy.

### SUMMARY OF THE INVENTION

In view of the above, the present invention is directed to design probes for detecting specific molecules on the surface of cell membrane based on the affinity between the specific molecules and the probes. Thus, the present invention provides a fast and accurate detection method as well as relevant cell detection chips and fabricating method thereof.

It is an object of the present invention to provide a method of fabricating cell detection chip and to fabricate a cell detection chip for recognizing normal cells and pathologically changed cells.

It is another object of the present invention to provide a cell detection chip having cell microarrays for simultaneously detecting different types of cells.

It is a further object of the present invention to provide a cell detection method for fast and accurate analysis of cell types (e.g., normal cells and pathologically changed cells) so as to provide appropriate treatments to a patient.

In accordance to the above objects and other advantages of the present invention, as broadly embodied and described herein, the present invention provides cell detection cell fabricating method. A plurality of probe molecules are designed, and there is specific affinity between each of the probe molecules and one of corresponding specific molecules on the cell surface. Wherein, the specific molecule is, for example, one from a group consisting of antibody and antigen. A spotting step is then preformed to spot these probe molecules respectively on the matrix. Especially, probe molecules can be designed to include simultaneously a plurality of quality control probes and/or a plurality of location indication probes, and such probe molecules can be synthesized and spotted like the aforementioned probe molecules.

This invention also provides a microarray detection chip applicable to detect specific molecules on cell surface, while the specific molecule is, for example, one from a group consisting of antibody and antigen. The microarray detection chip comprises a plurality of probes immobilized on a matrix, and each of the probes has special affinity with a specific molecule on the cell surface. In addition, the microarray detection chip can further comprise a plurality of quality control probes and/or a plurality of location indication probes.

This invention further provides a cell detecting method for detection of a plurality of specific molecules on the cell surface. A microarray detection chip as above mentioned is first provided. A biological sample is then obtained from a patient, wherein the sample includes a plurality of free cells. A cell reaction step is performed to react specific molecules on the surface of the free cells with the probes on the microarray detection chip. Next, a cell fixing step is performed to fix the free cells on the microarray detection chip. Then, an analysis step can be performed on the microarray detection chip.

Since the microarray detection chip of the invention has a plurality of probe molecules which has affinity respectively to specific molecules on the cell surface, the cell detection chip can be used to recognize different cells (e.g., normal cell and pathologically changed cell), and further to confirm the type of diseases that a patient suffers from, so as to provide a guidance on appropriate treatment for the patient.

It is to be understood that both the foregoing general description and the following detailed description are exemplary, and are intended to provide further explanation of the invention as claimed.

### BRIEF DESCRIPTION OF THE DRAWINGS

FIG. 1 is a flow chart illustrating a process of fabricating a cell detection chip according to one preferred embodiment of the present invention.

FIG. 2 is a flow chart illustrating steps of a cell detecting method according to one preferred embodiment of the present invention.

### DESCRIPTION OF THE PREFERRED EMBODIMENTS

The following description to the preferred embodiments of the present invention, as illustrated in the accompanied drawings, are set forth, for the purpose of explanation and not limitation, to provide a thorough understanding of the present invention.

Probe design is the focus of the fabrication of a microarray detection chip; the microarray detection chip can provide correct information only if the probe has special affinity with specific component in the sample to be detected. Based on such a concept, the present invention provides a method of fabricating microarray detection chip and a method of cell detecting. The following is an exemplary description on a method of fabricating a microarray detection chip for detecting acute myeloid leukemia (AML).

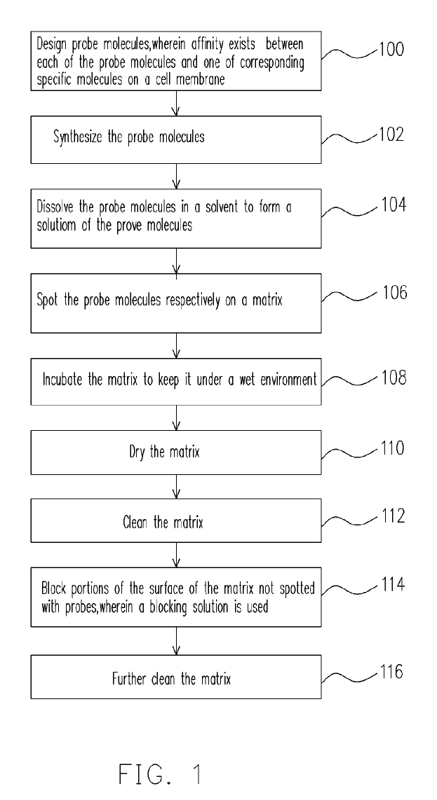

FIG. 1 illustrates a process of fabricating a cell detection chip according to one preferred embodiment of the present invention. A plurality of probe molecules are designed in accordance with a plurality of specific molecules on the cell surface, wherein each of the probe molecules has affinity to a specific molecule on the cell surface (step 100). Taking AML as an example, when a specific antigen of the disease bearing germs enters a patient's body, the patient's immune system will produce an antibody to resist the germs for destroying the germs. In the patient's body, there exist concurrently normal cells and pathologically changed cells, and there exist antigens and antibodies resisting the antigens on the surface of the pathologically changed cells. The AML can be classified into seven types based on the French-American-British (FAB) classification system. These seven types of AML and their corresponding antigens are listed in Table 1 as follows.

TABLE 1

| | Type of | | | | | | |
|---|---|---|---|---|---|---|---|
| Type of | acute myeloid leukemia (AML) | | | | | | |
| antigen | M1 | M2 | M3 | M4 | M5 | M6 | M7 |
| CD34 | W | W | | W | W | + | W |
| HLA-DR | + | + | | | | | T |
| CD13 | + | + | + | + | + | + | W |
| CD33 | + | + | + | + | + | + | W |
| CD15 | + | + | + | + | + | | |
| CD14 | | T | | + | + | | |
| CD11b | | T | | + | + | | |
| CD36 | | | | + | + | + | T |
| CD71 | | | | | | | |
| Glyco | | | | | | + | |
| CD41 | | | | | | + | |
| CD61 | | | | T | T | | + |
| Platelet | | | | T | T | | + |
| Ab | | | | | | | + |

Note:
W: weak reaction;
T: trace reaction.

As shown in Table 1, different types of AML show different antigen reactions on the surface of the pathologically changed cells. There is specificity between an antigen and an antibody, i.e., there exists affinity between a specific antigen and a specific antibody, and thus a specific antigen will combine only with a specific antibody. For designing probes to detect type of AML that a patient suffers from, probe molecules can be provided by employing antigens of CD34, HLA-DR, CD13, CD33, CD15, CD14, CD11b, CD36, CD71, Glyco, CD41, CD61, and Platelet Ab. In another preferred embodiment of the invention, probe molecules can be also provided by using the corresponding antibodies of the foregoing antigens to detect type of AML that a patient suffers from. In addition, in a still another preferred embodiment of the invention, probe molecules can be provided by using a combination of the foregoing antigens and their corresponding antibodies to detect type of AML.

It is should be noted that, as shown in Table 1, the weak reaction (W) or trace reaction (T) of antigens on the cell surface can be taken only as a minor indication to diagnose the type of AML that a patient suffers from. In other words, “+” is taken as a major indication, while “W” or “T” is merely taken as a minor indication for the diagnosis of AML. Using types M1 and M2 as examples, on the pathologically changed cells there exist antigens of CD34, HLA-DR, CD13, Cd33 and CD15. Types of M1 and M2 of AML can be recognized based on whether small amount of CD14 and CD11b antigens exists on the surface of pathologically changed cells.

A probe synthesizing step is then preformed to form a plurality of probes (step 102). During the synthesis of the probes, the compounds of probes can be modified, so that in the subsequent spot step, the compounds of the probes can bind via covalent force with functional groups on the surface of a matrix so as to be immobilized on the matrix.

The probes are dissolved in a solvent to form a plurality of corresponding probe solutions (step 104). Wherein, the probe spotting solvent is an aqueous phosphate buffer solution (PBS) having pH of 9.5 and concentration of, for example, 150 mg/L.

A spotting step is than performed to spot the probe spotting solution onto the matrix (step 106). Wherein, the radius of the probe spots is, for example, between 50 and 500 μm depending on different spotting methods. Note that under different circumstances for probe spotting on the matrix, each probe spotting solution is not limited to be used for a single spotting process. Since the matrix surface area is relatively large comparing to the amount of the probe spotting solution, there can be distributed tens or even hundreds spots of probe spotting solutions. Glass can be used as the matrix, no special facilities are required in the subsequent detecting steps, and thus merely a regular or existing optical microscope is needed to analyze the detection results.

An incubating step is performed for keeping the matrix under a wet environment (step 108). Wherein, the incubating step is performed under conditions of, for example, at 37° C. for 45 min.

A drying step is performed to dry the matrix (step 110). Wherein, the drying step is performed under conditions of, for example, at 40° C. for 2 hrs.

A matrix cleaning step is performed to clean the matrix (step 112). The cleaning step includes prior and subsequent cleaning steps and a drying step. Wherein, the cleaning liquid used in the cleaning step consists of buffer and de-ionized water, while the buffer includes PBS and 0.1% sodium dodecyl sulfate (SDS).

A blocking step is performed by using a blocking solution to block the surface of the matrix without probe spots (step 114). Wherein, the blocking solution is, for example, an aqueous solution of pH 7 comprising 1% bovine serum albumin (BSA) and 0.01 mol/L phosphate buffer (PB).

A further matrix cleaning step is performed to clean the matrix (step 116). The cleaning step includes prior and subsequent cleaning steps and a drying step. Wherein, the matrix cleaning step can be repeated for multiple times till the matrix is thoroughly cleaned. Wherein, the cleaning liquid used in the cleaning step is, for example, deionized water to wash out the excessive blocking solution. In one preferred embodiment of the invention, the matrix cleaning step is repeated three times.

The above-described method can be employed to fabricate a detection chip. Since the detection chip has a plurality of probe molecules which have affinity respectively to specific molecules on the cell surface, the detection chip can be used to recognize different cells (e.g., normal cell and pathologically changed cell), and further to confirm the type of AML, so as to provide a guidance on appropriate treatment for the patient.

It should be noted that the aforementioned step of designing probe molecule (step 100) could further be performed to design a plurality of quality control probes. In the subsequent probe synthesizing step through spotting step (step 102 to step 116), quality control probes are synthesized and immobilized on the matrix so that the resultant cell detection chip has quality control probes thereon. The quality control probes are correlated with specific components in the sample, and the correlations can be used to determine whether the sample detection is effective so as to avoid occurrence of detection errors.

In addition to design quality control probes, the aforementioned probe designing step (step 100) could be performed to design a plurality of location indication probes. Similarly, in the subsequent probe synthesizing step through spotting step (step 102 to step 116), location indication probes are synthesized and immobilized on the matrix so that the resultant cell detection chip has location indication probes thereon. The location indication probes are employed as location indicators on the microarray chips, for indicating relative locations of the probes on the microarray chips, so as to avoid occurrence of detection errors.

The cell detection chip prepared through the above methods comprises a plurality of probes immobilized on the matrix, and each probe has affinity to a specific molecule on the cell surface. Wherein, the probes are, for example, one of the types consisted of antibody and antigen.

To detect AML for example, the probes are, as shown in Table 1, antigens, antibodies corresponding to the antigens, or ones from the groups consisting of the antigens and antibodies. Further, type of antigens, of antibodies corresponding to the antigens, or of antigens and antibodies, as shown in Table 1, can occur for multiple times, such that a microarray detection chip can be formed with tens or even hundreds of probes thereon.

As the probes are correlated with the antibodies or antigens presented on the surface of cell membranes in an AML patient's body, the detection chip can be used to detect the type of AML that a patient suffered from.

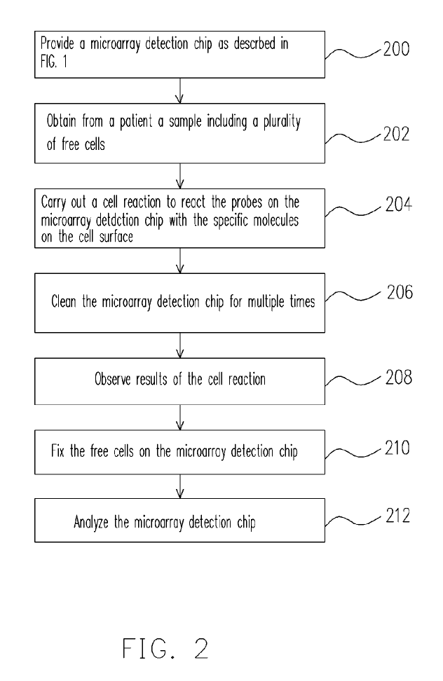

In the following, a cell detecting method for detecting the type of AML by using the foregoing microarray detection chips is described. FIG. 2 illustrates steps of a cell detecting method according to one preferred embodiment of the present invention.

Referring to FIG. 2, a microarray detection chip is first provided (step 200). The microarray detection chip is, for example, a microarray detection chip obtained by the aforementioned method, and, on the microarray detection chip there exist a plurality of specific probes capable of detecting the type of antibodies or antigens on the surface of cell membrane. In a preferred embodiment of the present invention, on a microarray detection chip further immobilized quality control probes and/or location indication probes, in addition to the specific probes capable of detecting the type of antibodies or antigens on the surface of cell membrane.

A biological sample is then obtained from a patient, and such sample includes a plurality of free cells (step 202). Wherein, the sample is, for example, pleural fluid, ascites, urine, or blood. Subsequently, the sample needs to be rinsed with an aqueous PBS solution for multiple times. In a preferred embodiment of the present invention, the sample is rinsed with an aqueous PBS solution for 3 times while each rinse takes about 5 min and the adjusted cell concentration is 10⁸/L.

A cell reaction step is then performed to react the specific molecules on the surface of cell membrane of the free cells with the probes on the microarray chip (step 204). In the step 204, if, on the surface of cell membrane of the cells in a biological sample obtained from a patient, there exist antibodies or antigens correlated with the probes, the cells will be caught onto the detection chip through the affinity (e.g., van der Waals' force) between the probes and the antibodies or between the probes and the antigens.

A cleaning step is performed for multiple times to clean the microarray chip (step 206). Wherein, cleaning liquid used in the cleaning step is, for example, aqueous PBS solution at room temperature. Through the cleaning step, cells not reacted with the probes (in step 204) are washed out and cells reacted with the probes remain.

An observation step (step 208) is then performed to observe reaction results of the cells. Wherein, a regular optical microscope can be used to observe and confirm the preliminary reaction results.

A cell fixing step (step 210) is performed to fix the free cells onto the microarray detection chip. Wherein, 2.5% glutaraldehyde is used for 5 min for example.

An analysis step (step 212) is performed to analyze the microarray chip. In this invention, the detail procedure in the analysis step may vary with different requirements, but generally there are no specific restrictions; i.e., different procedures can be used depending on user's requirements. Thus, the analysis step can be carried out via rule staining, immunohistochemistry staining, in-situ hybridization, cell culture, drug analysis, or other appropriate analytical methods. Of course, different preliminary treatments on the detection chip may be necessary for certain analysis methods. Such preliminary treatments are known by one of ordinary skill in the art and thus are not described in detail here. In the following, rule staining method is described as an example. After the completion of the cell fixing step (step 210), Wright's stain is used for 15 min for rule staining. The microarray chip is then dyed through a process of three-color fluorescent dye. A fluorescence scanner is used to scan the microarray chip for obtaining detection data. The analysis results on the microarray detection chip are output by employing analysis software compatible with the fluorescence scanner, so as to recognize the type of AML that a patient suffers from. Wherein, the scanner is provided by, for example, Genomic Solutions.

Even though, in this embodiment, detection of the type of AML is used to describe the invention, the invention is not so limited. The invention is applicable in other fields. In another preferred embodiment of the invention, glyco-protein antibodies on the cell surface are used as the probes to analyze the types of malignancy and the relevant drug resistance. In still another preferred embodiment, in-situ carcinoma gene protein antibodies are used as the probes to analyze cell types and further to diagnose cancers, such as breast cancer, ovarian cancer, lung cancer, gastric cancer, esophagus cancer, cervical cancer, salivary cancer, or bladder cancer. In still another preferred embodiment, cyto skeleton protein antibodies are used as the probes to analyze mechanism of malignancy transfer and infiltration. In still another preferred embodiment, surface antigens of while cells are used as the probes to analyze cell type and malignance in blood and various body fluids. In still another preferred embodiment, receptor antibodies on the cell surface are used as the probes to analyze different cell types. In further a preferred embodiment, target antibodies of the malignance cell membrane are used as the probes to analyze tumor types.

To sum up, this invention has at least the following advantages: First, the methods of this invention can be employed to fabricate cell detection chips. Since the microarray detection chip of the invention has a plurality of probe molecules which has affinity respectively to specific molecules on the cell surface, the cell detection chip can be used to recognize different cells (e.g., normal cell and pathologically changed cell), and further to confirm the type of diseases that a patient suffers from, so as to provide a guidance on appropriate treatment for the patient. Second, large amount of accurate analytical results can be obtained through cell detection by the use of microarray detection chip of this invention. Third, the microarray detection chip on this invention can be used in researches on cell taxonomy, cell physiology, cell biology, cell toxicology, pharmacology and cell development.

It will be apparent to those skilled in the art that various modifications and variations can be made to the structure of the present invention without departing from the scope or spirit of the invention. In view of the foregoing, it is intended that the present invention covers modifications and variations of this invention provided they fall within the scope of the following claims and their equivalents.

1. A microarray detection chip applicable to detecting a plurality of specific molecules on a cell surface, comprising:
a plurality of probes immobilized on a matrix, wherein an affinity exists between each of the probes and one of corresponding specific molecules on the cell surface.

2. The microarray detection chip as in claim 1, wherein the specific molecules comprise at least one from a group consisting of antibodies and antigens.

3. The microarray detection chip as in claim 1, further comprising a plurality of quality control probes immobilized on the matrix.

4. The microarray detection chip as in claim 1, further comprising a plurality of location indication probes immobilized on the matrix.
